(19) United States
(12) Patent Application Publication
(10) Pub. No.: US 2025/0349003 A1
(43) Pub. Date: Nov. 13, 2025

(54) SYSTEMS AND METHODS FOR FACILITATING SCREENING OF BRAIN AGE USING CT IMAGERY

(71) Applicants: THE UAB RESEARCH FOUNDATION, Birmingham, AL (US); BODY CHECK, LLC, Vestavia Hills, AL (US)

(72) Inventors: Andrew Dennis SMITH, Hoover, AL (US); Steven Adam ROTHENBERG, Vestavia Hills, AL (US); Yujan SHRESTHA, Castle Pines, CO (US); Jose Angelo Udal PERUCHO, Birmingham, AL (US); Yu-Hua FANG, Vestavia, AL (US)

(21) Appl. No.: 19/201,380

(22) Filed: May 7, 2025

Related U.S. Application Data
(60) Provisional application No. 63/644,659, filed on May 9, 2024.

(51) Int. Cl. G06T 7/00; G06T 7/62; G16H 50/30

(52) U.S. Cl. CPC G06T 7/0012; G06T 7/62; G16H 50/30; G06T 2207/10081; G06T 2207/20081; G06T 2207/30016

(57) ABSTRACT

A computer-implemented method for assessing brain age comprises (i) obtaining a set of computed tomography (CT) images, the set of CT images capturing at least a portion of a brain of a patient, the set of CT images being captured for a purpose independent of assessing brain age; (ii) using the set of CT images as an input to an artificial intelligence (AI) module configured to determine a brain measurement based on CT image set input; (iii) obtaining a brain measurement output based on output of the AI module; (iv) using the brain measurement output to calculate a set of quantitative metrics associated with the brain of the patient; and (v) using the set of quantitative metrics and a chronologic age of the patient to calculate a brain age score of the brain of the patient.

### CROSS-REFERENCE TO RELATED APPLICATIONS

This application claims priority to and the benefit of U.S. Provisional Application No. 63/644,659, filed on May 9, 2024, and entitled “SYSTEMS AND METHODS FOR FACILITATING SCREENING OF BRAIN AGE USING CT IMAGERY”, the entirety of which is incorporated herein by reference for all purposes.

### BACKGROUND

Many individuals experience brain pathologies that remain undetected until the individual undergoes screening and/or testing that is tailored to detect such brain pathologies. Such brain pathologies can be asymptomatic (e.g., in early stages), which can cause a delay between the initial development of a brain pathology in an individual and the performance of screening and/or testing to diagnose the brain pathology. Such delay can allow brain pathologies to advance, deepen, exacerbate, and/or aggravate before diagnosis and/or treatment can begin, which can cause undesirable outcomes for patients. Individuals often receive computed tomography (CT) exams in order to diagnose a brain pathology, but often receive a CT exam after the patient is symptomatic and the brain pathology has progressed.

Accordingly, there are a number of disadvantages with current methods for screening for brain pathologies that may be addressed.

The subject matter claimed herein is not limited to embodiments that solve any disadvantages or that operate only in environments such as those described above. Rather, this background is only provided to illustrate one example problem space where some embodiments described herein may be practiced.

### BRIEF DESCRIPTION OF THE DRAWINGS

FIG. 1 illustrates an example computer system that may comprise or implement one or more embodiments of the present disclosure;

FIG. 2 illustrates an example flow diagram depicting acts associated with assessment of brain age using a set of quantitative metrics;

FIG. 3 illustrates an example flow diagram depicting acts associated with facilitating opportunistic assessment of brain age;

FIG. 4 illustrates an example flow diagram depicting acts associated with facilitating assessment of brain age using a normative index of quantitative metrics;

FIGS. 5A through 5E illustrate example CT images and brain measurements associated with assessment of brain age; and

FIG. 6 illustrates example components of a report summarizing findings related to assessment of brain age and associated clinical conditions.

### DETAILED DESCRIPTION

Before describing various embodiments of the present disclosure in detail, it is to be understood that this disclosure is not limited to the parameters of the particular example systems, methods, apparatus, products, processes, and/or kits, which may, of course, vary. Thus, while certain embodiments of the present disclosure will be described in detail, with reference to specific configurations, parameters, components, elements, etc., the descriptions are illustrative and are not to be construed as limiting the scope of the claimed invention. In addition, any headings used herein are for organizational purposes only, and the terminology used herein is for the purpose of describing the embodiments. Neither are meant to be used to limit the scope of the description or the claims.

Embodiments of the present disclosure are directed to systems and methods for facilitating opportunistic screening for brain pathologies using CT imaging data, as well as systems and methods for facilitating extraction of quantitative information about the cognitive health of a patient that is not routinely extracted from CT imaging data.

As used herein, the term “physician” generally refers to a medical doctor, or a specialized medical doctor, such as a radiologist, primary care physician, neurologist, or other medical doctor. This term may include any other medical professional, practitioner, or clinician, including any licensed medical professional or other healthcare practitioners, such as a physician's assistant, a nurse, a veterinarian (such as, for example, when the patient is a non-human animal), etc.

As used herein, the term “patient” generally refers to any human or animal, for example a mammal, under the care of a physician, as that term is defined herein, with typical reference to humans who have undergone brain imaging, and particularly those who have undergone a CT scan of the head. Such humans may include research participants, individuals under the care of a medical professional, and/or others. For purposes of the present application, a “patient” may be interchangeable with an “individual” or “person.” In some embodiments, the individual is a human patient.

### Overview of Disclosed Embodiments

As used herein, opportunistic screening refers to passive screening for one or more particular brain pathologies and/or quantitative information about the cognitive health of a patient using medical imagery and/or reports obtained for a purpose that is independent of screening for the particular brain pathology and/or quantitative information. For instance, a physician may order a brain scan for a patient after an accident, (e.g., a car accident that results in head trauma) and images of the patient acquired pursuant to the brain scan may be analyzed/screened (e.g., in opportunistic fashion) to detect other brain pathologies (e.g., dementia, Alzheimer's disease, stroke, hydrocephalus, and/or others) and/or quantitative information about the cognitive health of the patient. The quantitative information about the cognitive health of the patient may comprise a set of quantitative metrics indicative of the general cognitive health of a patient. The set of quantitative metrics may be used to calculate other indicators of cognitive health such as a brain age of a patient.

As used herein, the term “brain age” refers to a metric that indicates the estimated age of a brain of a person based at least in part on additional factors/information beyond the chronological age of the person. In one example, brain age may be calculated using physical properties of the brain of a patient and comparing the physical properties of the brain of the patient to the physical properties that are indicative of a normal brain at a particular chronological age from birth. For instance, patients with abnormal levels of brain atrophy (e.g., volume loss) and/or an abnormally low brain density would have a higher brain age than patients with normal levels of brain atrophy and/or a normal brain density.

In some embodiments, a computer-implemented method for assessing the brain age of a patient includes obtaining a set of computed tomography (CT) images. The set of CT images captures at least a portion of a brain of a patient. The method further includes processing the set of CT images using one or more artificial intelligence (AI) modules to obtain a set of quantitative metrics associated with the brain of the patient, the set of quantitative metrics can comprise total brain volume, ventricular volume, intracranial extra-axial cerebral spinal fluid (CSF) volume, and/or atherosclerotic calcifications. The method further includes using the set of quantitative metrics to determine a brain age score for the brain of the patient.

In some embodiments, a computer-implemented method for facilitating opportunistic screening for brain pathologies and/or quantitative information about the cognitive health of a patient includes obtaining a set of CT images. The set of CT images captures at least a portion of a brain of a patient, and the set of CT images is captured for a purpose independent of assessing a particular brain pathology. The method further includes using the set of CT images as an input to an AI module configured to determine a brain measurement based on CT image set input. The method also includes obtaining brain measurement output generated using the AI module and, based on the brain measurement output, calculating a set of quantitative metrics associated with the brain of a patient. The method further includes using the set of quantitative metrics and a chronologic age of the patient to calculate a brain age score of the brain of the patient.

In some embodiments, a computer-implemented method for facilitating opportunistic screening for brain pathologies and/or quantitative information about the cognitive health of a patient includes obtaining a set of CT images. The set of CT images captures at least a portion of the brain of a patient, and the set of CT images is captured for a purpose independent of assessing a particular brain pathology. The method further includes using the set of CT images as an input to an AI module configured to generate a set of quantitative metrics associated with the brain of a patient. The set of quantitative metrics comprises a set of values for one or more brain age parameters, the one or more brain age parameters comprising one or more of: total brain volume, total brain density, ventricular volume, extra-axial CSF volume and atherosclerotic calcifications. The method further includes comparing the set of quantitative metrics to a normative index of head CT data to determine a comparative output. The normative index comprises a set of normative values for the one or more brain age parameters. The set of normative values is associated with healthy brains at different chronological ages. The method further includes using the comparative output to calculate a brain age score of the brain of the patient.

Those skilled in the art will appreciate, in view of the present disclosure, that at least some of the disclosed embodiments may address shortcomings and/or deficiencies associated with conventional processing and/or use of head CT exams. For example, there is quantitative information in head CT images that is not routinely extracted and therefore goes underutilized in the diagnosis of brain pathologies, and/or overall cognitive health. Some of the disclosed embodiments can enable extraction of such information, and the information may be used to detect asymptomatic brain pathologies, predict the development of future brain pathologies, and/or assess the general cognitive health of a patient. For example, at least some embodiments of the present disclosure use quantitative information extracted from head CT images to calculate a brain age score for the brain of a patient. The brain age score may be used by a physician as a screening test for existing brain pathologies in a patient, such as mild cognitive impairment, dementia, Alzheimer's disease, stroke, hydrocephalus, normal pressure hydrocephalus, alcoholism, drug toxicity, congenital abnormalities, brain death, and behavioral, neurologic, and psychiatric disorders. Furthermore, a brain age score may be used by a physician to predict the onset of brain pathologies in a patient. Additionally, or alternatively, a brain age score may be used by a physician as an indicator of general cognitive health or as part of a routine health checkup. For example, extracting metrics such as brain volume, density, and/or estimated brain age from a head CT image may be used to screen healthy individuals (i.e., patients who are not experiencing symptoms of a particular brain pathology) for brain pathologies that have not yet manifested, providing physicians with an opportunity to detect pathologies early, possibly improving patient outcomes.

At least some embodiments of the present disclosure can be used to extract quantitative information from head CT images to estimate brain age, assess overall cognitive health, and/or detect brain pathologies from previously acquired head CT exams. For example, a head CT exam may have been acquired as part of clinical care, screening, or research.

At least some embodiments of the present disclosure may utilize a normative index comprising quantitative information extracted from head CT scans, wherein the quantitative information is extracted from patients with healthy brains at various chronological ages. The normative index can be generated using at least some of the embodiments of the present disclosure. Furthermore, the normative index may comprise a set of CT images of healthy brains at different chronological ages, wherein some embodiments may enable a physician to compare a head CT from one patient to preexisting head CT images from healthy patients. Additionally, or alternatively, some embodiments may enable a physician to compare segmented structures with a head CT image from one patient to segmented structures within preexisting head CT images from healthy patients. These comparative techniques may be used to estimate a brain age score for the brain of a patient, screen a patient for brain pathologies, and/or assess the overall cognitive health of a patient.

### Exemplary Systems and Methods for Brain Pathologies and/or Cognitive Health Information

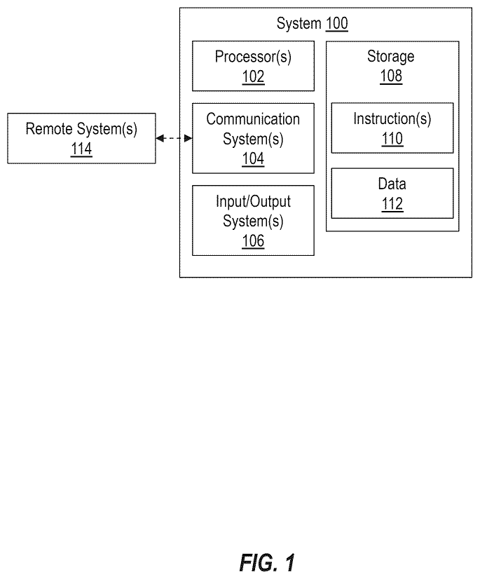

Having described some of the various high-level features and benefits of the disclosed embodiments, attention will now be directed to FIGS. 1 through 6. These Figures illustrate various conceptual representations, architectures, methods, and/or supporting illustrations related to the disclosed embodiments.

FIG. 1 illustrates an example computer system that may comprise or implement one or more embodiments of the present disclosure. As is illustrated in FIG. 1, the computer system 100 includes processor(s) 102, communication system(s) 104, I/O system(s) 106, and storage 108. Although FIG. 1 illustrates the computer system 100 as including particular components, it will be appreciated, in view of the present disclosure, that a computer system 100 may comprise any number of additional or alternative components.

The processor(s) 102 may comprise one or more sets of electronic circuitry that include any number of logic units, registers, and/or control units to facilitate the execution of computer-readable instructions (e.g., instructions that form a computer program). Such computer-readable instructions may be stored within storage 108. The storage 108 may comprise physical system memory or computer-readable recording media and may be volatile, non-volatile, or some combination thereof. Furthermore, storage 108 may comprise local storage, remote storage, or some combination thereof. Additional details related to processors (e.g., processor(s) 102) and computer storage media (e.g., storage 108) will be provided hereinafter.

As used herein, processor(s) 102 may comprise or be configurable to execute any combination of software and/or hardware components that are operable to facilitate processing using machine learning models or other artificial intelligence-based structures/architectures. For example, processor(s) 102 may comprise and/or utilize hardware components or computer-executable instructions operable to carry out function blocks and/or processing layers configured in the form of, by way of non-limiting example, single-layer neural networks, feed forward neural networks, radial basis function networks, deep feed-forward networks, deep learning modules, recurrent neural networks, long-short term memory (LSTM) networks, gated recurrent units, autoencoder neural networks, variational autoencoders, denoising autoencoders, sparse autoencoders, Markov chains, Hopfield neural networks, Boltzmann machine networks, restricted Boltzmann machine networks, deep belief networks, deep convolutional networks (or convolutional neural networks), deconvolutional neural networks, deep convolutional inverse graphics networks, generative adversarial networks, liquid state machines, extreme learning machines, echo state networks, deep residual networks, Kohonen networks, support vector machines, random forest models, neural Turing machines, and/or others.

As will be described in more detail, the processor(s) 102 may be configured to execute instructions 110 stored within storage 108 to perform certain actions associated with facilitating opportunistic screening for brain pathologies and/or cognitive health information. The actions may rely at least in part on data 112 stored on storage 108 in a volatile or non-volatile manner (e.g., one or more sets of CT images). In some instances, the actions may rely at least in part on communication system(s) 104 for receiving data from remote system(s) 114, which may include, for example, other computer systems or computing devices, medical imaging devices/systems, and/or others.

The communications system(s) 104 may comprise any combination of software or hardware components that are operable to facilitate communication between on-system components/devices and/or with off-system components/devices. For example, the communications system(s) 104 may comprise ports, buses, or other physical connection apparatuses for communicating with other devices/components (e.g., USB port, SD card reader, and/or other apparatus). Additionally, or alternatively, the communications system(s) 104 may comprise systems/components operable to communicate wirelessly with external systems and/or devices through any suitable communication channel(s), such as, by way of non-limiting example, Bluetooth, ultra-wideband, WLAN, infrared communication, and/or others.

Furthermore, in some instances, the actions that are executable by the processor(s) 102 may rely at least in part on I/O system(s) 106 for receiving user input from one or more users. I/O system(s) 106 may include any type of input or output device such as, by way of non-limiting example, a touch screen, a display, a mouse, a keyboard, a controller, and/or others, without limitation.

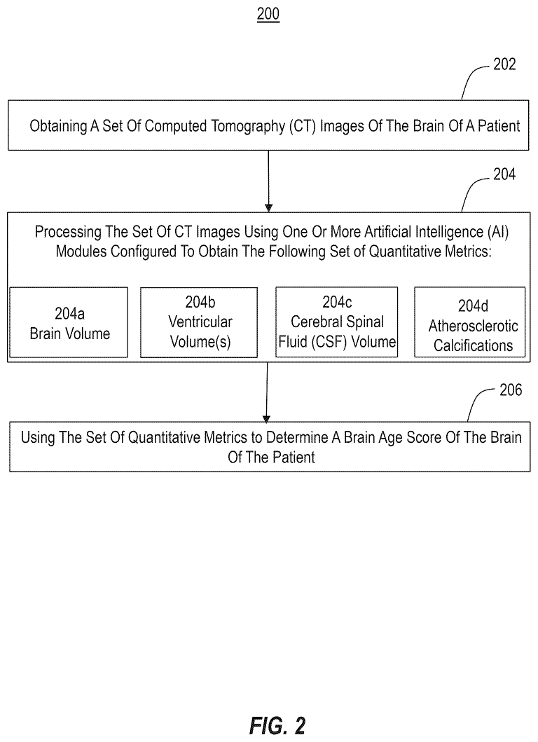

Some embodiments of the present disclosure can also be described in terms of acts (e.g., acts of a method) for accomplishing a particular result. Along these lines, FIGS. 2 through 4 illustrate example flow diagrams 200, 300, and 400 respectively, depicting acts associated with facilitating screening for brain pathologies and/or cognitive health information. Although the acts shown in flow diagrams 200, 300, and 400 may be illustrated and/or discussed in a certain order, no particular ordering is required unless specifically stated or required because an act is dependent on another act being completed prior to the act being performed. Furthermore, it should be noted that, in some implementations, not all acts represented in flow diagrams 200, 300, and 400 are essential for facilitating screening for brain pathologies and/or cognitive health information.

In some instances, the various acts disclosed herein are performed using a computer system 100. For instance, code for configuring the computer system 100 to perform the various acts disclosed herein may be stored as instructions 110 on storage 108, and such instructions 110 may be executable by the processor(s) 102 (and/or other components) to facilitate carrying out of the various acts.

Act 202 of flow diagram 200 includes obtaining a set of computed tomography (CT) images of the brain of a patient. The set of CT images may comprise contrast-enhanced, non-contrast (or nonenhanced), high resolution, and/or any other format of CT images. The set of CT images comprises images obtained from CT scans taken of the head of a patient. Accordingly, the set of CT images captures at least a portion of the brain of a patient whose body is represented in the set of CT images. In some instances, although a set of CT images may not provide a representation of the entire brain of a patient, a set of CT images may capture one or more representations of key brain structures (e.g., the entire brain, the ventricular spaces, the sulci, the skull, atherosclerotic calcifications, extra axial CSF spaces, the cortex, white matter regions, grey matter regions, and/or others) that may be used to detect brain pathologies and/or assess the cognitive health of a patient. Accordingly, a set of CT images may include one or more cross-sectional images that provide a largest possible cross-sectional representation of one or more key brain structures.

In some embodiments, the set of CT images can be restricted to certain attenuation thresholds to evaluate key metrics related to the brain of a patient. For example, the set of CT images can be restricted to CT images with attenuation values between −10 HU and +99 HU (or +40 HU to +99 HU) in order to exclude undesired structures present in the set of CT images such as gas, fluid, bone, or metal. The set of CT images can be restricted to CT images with attenuation values with alternative ranges as described hereinafter. The set of CT images can be selected to omit soft tissue structures outside of the skull. The set of CT images may capture the intracalvarial compartment (e.g., structures inside the skull).

Act 204 of flow diagram 200 includes using the set of CT images as an input to an artificial intelligence (AI) module configured to process the set of CT images to generate a set of quantitative metrics. FIG. 2 depicts example quantitative metrics associated with act 204, including brain volume 204a (e.g., segmental brain volumes or total brain volume), ventricular volume(s) 204b, cerebral spinal fluid (CSF) volume 204c (e.g., extra-axial CSF volumes), and atherosclerotic calcifications 204d (e.g., carotid atherosclerotic calcifications). Other quantitative metrics for key brain structures may be determined in accordance with the present disclosure (e.g., area, volume, distances, and/or other measurements associated with other types of brain structures). As described herein, the set of quantitative metrics can be used to assess the overall cognitive health of a patient, diagnose one or more brain pathologies, and/or assign a brain age score to a patient. The AI module can be configured to obtain additional quantitative metrics, which will be described hereinafter. One will appreciate, in view of the present disclosure, that the AI module may take on any suitable form and can include any suitable components for determining quantitative output based on CT image set input, such as, by way of non-limiting example, convolutional neural networks (CNNs), object detection models, semantic segmentation models, instance segmentation models, deep metric learning, regression models, combinations thereof, and/or others. One will appreciate, in view of the present disclosure, that the AI module(s) for determining the quantitative metrics in accordance with act 204 can include one or more additional pre-processing modules or post-processing modules (whether such additional modules are AI-based or not). By way of illustrative example, the AI module(s) can be configured to provide segmentation/mask output, and one or more post-processing modules may be used to measure/determine the quantitative metrics based on the segmentation/mask output.

In some embodiments, the AI module comprises one or more machine learning modules that is/are configured/trained to identify a subset of CT images from CT image set input. The subset of CT images can include one or more CT images that provide a largest representation of one or more key brain structures of a patient represented in the CT image set input (e.g., key brain structures of the patient associated with the set of CT images described with reference to act 202). Accordingly, the subset of CT images may provide a basis for determining a measurement associated with one or more of the key brain structures represented in the CT image set input. The AI module(s) may be trained on a training dataset including input data comprising CT image sets. The training dataset may further comprise ground truth output, which may comprise tags indicating which CT image(s) of the different training input CT image sets provide(s) a representation of one or more key brain structures of a patient. In some instances, the AI module(s) are configured to segment each of the CT images to identify whether a brain structure is represented In each of the CT images, and, where a brain structure is detected, the AI module(s) may be configured to determine automated brain structure measurements associated with the brain structure. The CT image(s) providing a largest representation of the one or more key brain structures of the patient may thus be identified by comparing the automated brain measurements obtained by the AI module(s). Appropriate training data may be utilized to configure the AI module(s) for such purposes (e.g., CT image input and ground truth tags indicating whether a brain or key brain structure is present in the CT image and/or indicating measurements for the brain or key brain structure). The quantitative metrics may be determined based on the automated brain measurements, such as via direct computation (e.g., length measurements, area measurements, pixel counting, etc.) and/or further AI processing.

In some embodiments, the AI module(s) may be configured to provide a volume measurement of the entire brain and/or a portion/aspect thereof to facilitate assessing the cognitive health of a patient. For example, the AI module(s) may be trained/configured to measure the total brain volume of a particular patient, and/or the volume of individual key brain structures. Additionally, or alternatively, the AI module(s) may be directed to provide a density measurement of the entire brain, and/or the density of individual key structures.

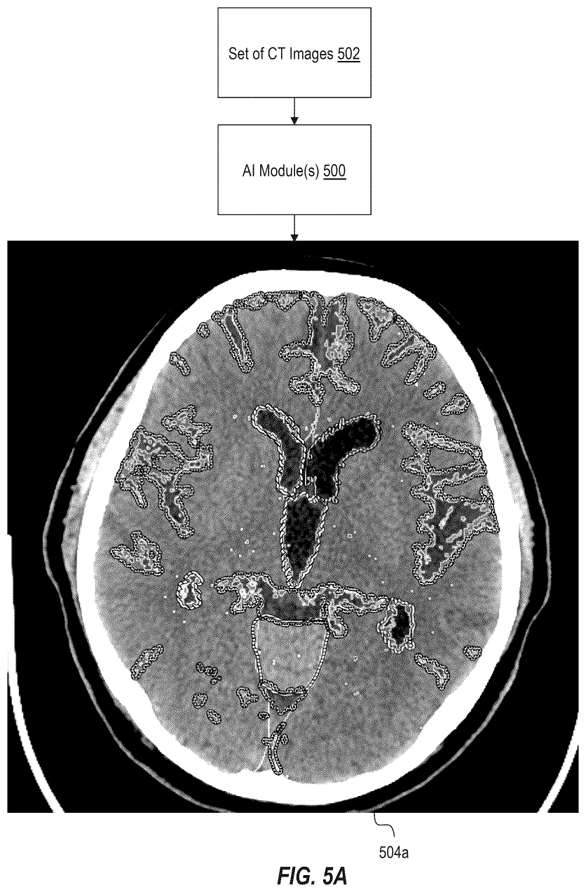

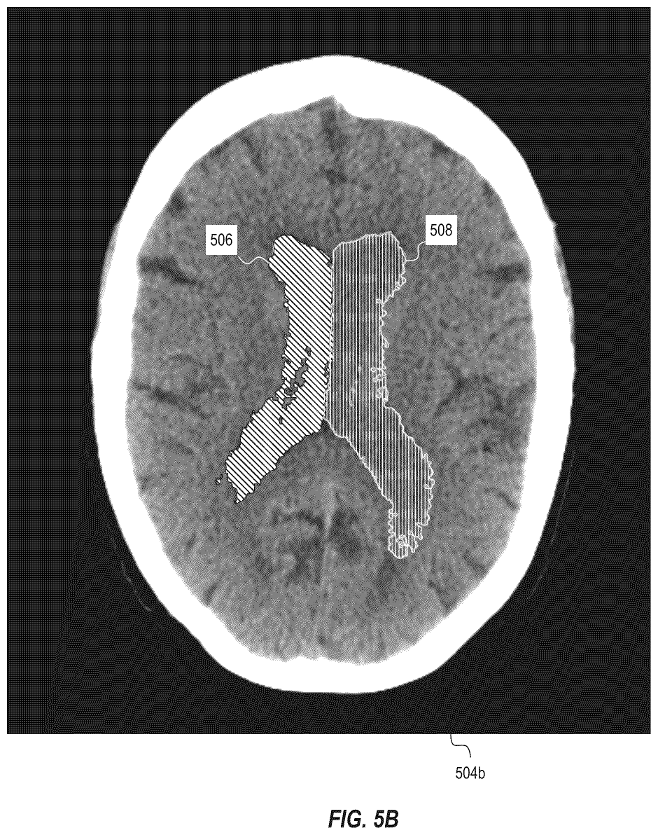

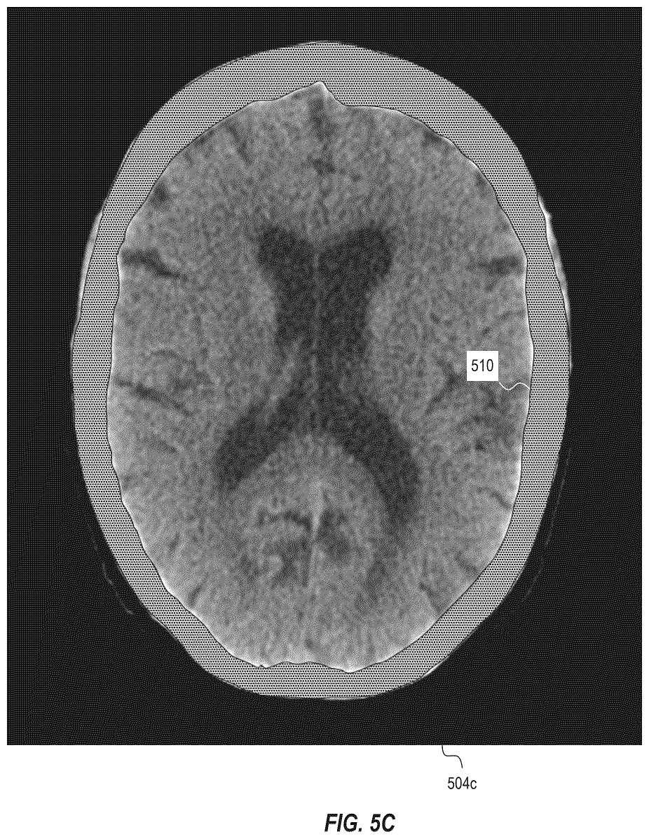

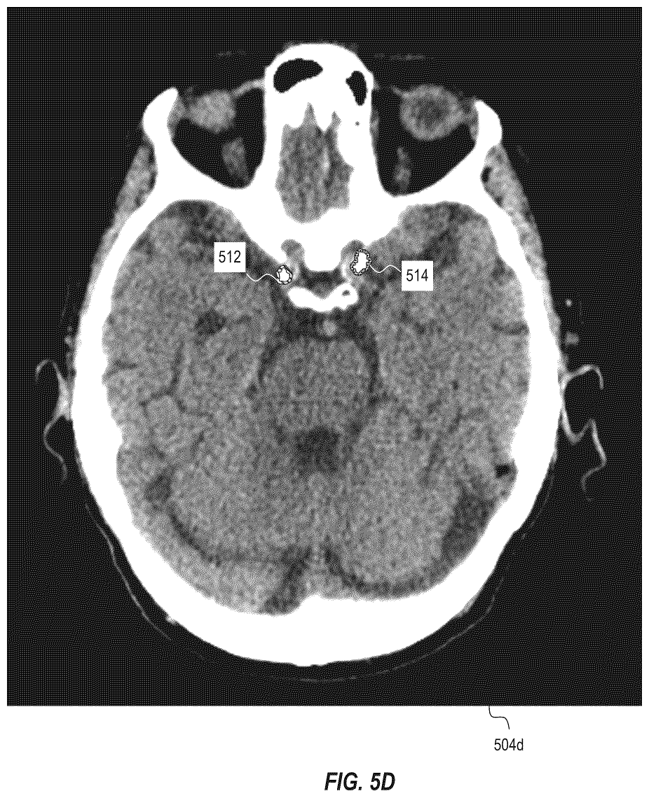

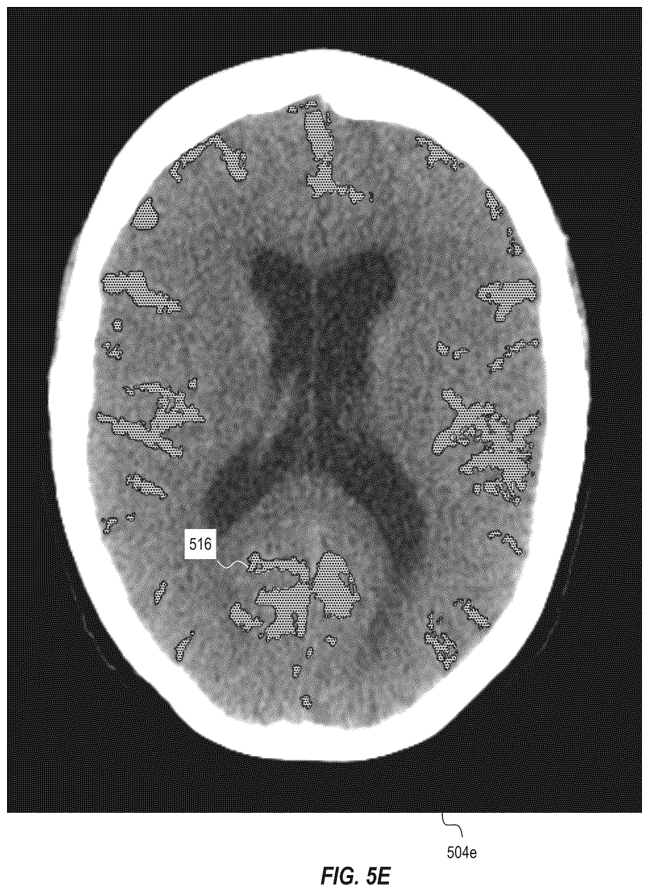

FIG. 5A illustrates a set of CT images 502 being provided as input to AI module(s) 500. The set of CT images 502 may correspond to the set of CT images discussed hereinabove with reference to acts 202 and 204 of flow diagram 200. Similarly, the AI module(s) 500 may correspond to the AI module(s) discussed hereinabove with reference to act 204 of flow diagram 200. The AI module(s) 500 can be configured to identify one or more CT images of an input set of CT images that provides a (largest) representation of patient's brain and/or key brain structures of a patient's brain. FIG. 5A illustrates a CT image 504a, which may be identified utilizing the AI module(s) 500 as depicting a representation of a patient's brain and/or one or more key brain structures of a patient's brain. CT image 504a comprises multiple overlays illustrating an identification of multiple key brain structures within a patient's brain (denoted by different line types). The overlays shown in CT image 504a can represent output or intermediate output of the AI module(s) 500. The AI module(s) 500 can be configured to produce a set of quantitative metrics associated with each overlaid region including volume and density measurements (and/or others as described hereinbelow). In some instances, the measurements associated with individual brain structures can be aggregated or combined (e.g., by addition and/or subtraction) to obtain composite brain metrics. For instance, total brain density and/or total brain volume (e.g., total brain volume 204a) may be obtained by aggregating measurements of individual brain structures or regions (potentially across multiple CT images/slices). As discussed hereinabove, such measurements may be obtained automatically utilizing the AI module(s) 500.

Referring again to FIG. 2, the AI module(s) of act 204 may be configured to generate quantitative metrics related to ventricular spaces of the brain of a patient (e.g., ventricular volume 204b) including the left ventricle, right ventricle, and/or the 4^{th} ventricle. The AI module(s) may be configured to measure one or more ventricular spaces on one axial slice or on multiple axial slices (e.g., one or more slices where the representation of a desired ventricular space is the largest in size). The AI module(s) may provide volume measurements of a particular ventricular space including right ventricle volume, left ventricle volume, and/or 4^{th} ventricle volume (e.g., by aggregating ventricular area measurements from multiple slices). Additionally, or alternatively, the AI module(s) may be directed to provide a density measurement of one or more ventricular spaces, including right ventricle density and/or left ventricle density.

By way of illustration, attention is directed to FIG. 5B, which shows the CT image 504b, comprising a representation of the brain of a patient with an overlay (or mask) illustrating an identification of the ventricular spaces of the patient's brain, including area 506 representing the right ventricle of a patient's brain and area 508 representing the left ventricle of a patient's brain. The overlay defining the areas 506 and/or 508 can comprise output or intermediate output of the AI module(s) 500. As discussed hereinabove, AI module(s) 500 may be used to automatically produce a set of quantitative metrics associated with the ventricular spaces of the patient's brain using CT image 504b.

Referring again to FIG. 5B, the AI module(s) may be configured to generate quantitative metrics related to the skull of a patient. The AI module(s) may be configured to measure the skull of a patient on one axial slice or on multiple axial slices. The AI module(s) may provide volume measurements of the skull, density measurements of the skull, and/or determine the skull area (which may rely on aggregation of metrics obtained from different image slices).

By way of illustration, attention is directed to FIG. 5C, which shows the CT image 504c, comprising a representation of the brain of a patient with an overlay illustrating an identification the skull of the patient, including area 510 representing the skull of a patient. The overlay defining the area 510 can comprise output or intermediate output of the AI module(s) 500. As discussed above, AI module(s) 500 may be used to automatically produce a set of quantitative metrics associated with the skull of the patient using CT image 504c.

Referring again to FIG. 2, the AI module(s) of act 204 may be directed to generate quantitative metrics related to atherosclerotic calcifications in the brain of a patient (e.g., atherosclerotic calcifications 204d). The AI module(s) may be configured to measure the extent of atherosclerotic calcification of the brain of a patient on one axial slice or on multiple axial slices (e.g., one or more slices where the representation of the atherosclerotic calcifications is the largest in size).

By way of illustration, attention is directed to FIG. 5D, which shows CT image 504d, comprising a representation of the brain of a patient with an overlay illustrating an identification of atherosclerotic calcifications in the brain of a patient, including areas 512 and 514 representing atherosclerotic calcifications in the brain of a patient. The overlay defining the areas 512 and/or 514 can comprise output or intermediate output of the AI module(s) 500. As discussed above, AI module(s) 500 may be used to automatically produce a set of quantitative metrics associated with the atherosclerotic calcifications in the brain of the patient using CT image 504d.

Referring again to FIG. 2, the AI module(s) of act 204 may be configured to generate quantitative metrics related to the extra-axial CSF spaces of the brain of a patient (e.g., CSF volume 204c). The AI module(s) may be configured to measure one or more extra-axial CSF spaces on one axial slice or on multiple axial slices (e.g., one or more slices where the representation of a desired ventricular space is the largest in size). The AI module(s) may provide a volume measurement of one or more extra-axial CSF spaces, which may be obtained by aggregating area measurements associated with multiple image slices. Additionally, or alternatively, the AI module(s) may be configured to provide a density measurement of one or more extra-axial CSF spaces.

By way of illustration, attention is directed to FIG. 5E, which shows the CT image 504e, comprising a representation of the brain of a patient with an overlay illustrating an identification of the extra-axial CSF spaces of the brain of a patient, including area 516 representing an extra-axial CSF space of the patient. The overlay defining the areas 516 and related areas can comprise output or intermediate output of the AI module(s) 500. As discussed above, AI module(s) 500 may be used to automatically produce a set of quantitative metrics associated with one or more extra-axial CSF spaces of the patient using CT image 504e.

The set of quantitative metrics generated using AI module(s) may be used to assess the overall cognitive health of a patient, diagnose one or more brain pathologies, and/or assign a patient with a brain age score. For example, because the human brain decreases in volume and density with age, patients displaying a lower brain volume and/or density may be assigned a higher brain age score. Other metrics can also be obtained using AI module(s) and can be used to assess the cognitive health of a patient. For example, low segmental brain volume, high percentage or volume of low density brain, low mean brain density, high CSF volume (including ventricles and sulci), low skull bone volume and density, low muscle volume and density, and high atherosclerotic calcifications are all associated with a higher brain age and poorer cognitive health. Such brain metrics can be used to predict brain age and cognitive health of a patient, and may be used to detect or predict various brain pathologies including: mild cognitive impairment, dementia, Alzheimer's disease, stroke, hydrocephalus, normal pressure hydrocephalus, alcoholism, drug toxicity, congenital abnormalities, brain death, and behavioral, neurologic, and psychiatric disorders.

As noted above, although act 204 of flow diagram 200 focuses, in at least some respects, on a specific set of quantitative metrics (i.e., total brain volume 204a, ventricular volume 204b, CSF volume 204c, and atherosclerotic calcifications 204d), additional quantitative metrics may be determined that relate to the cognitive or brain health of a patient.

In some embodiments, the set of quantitative metrics described in act 204 may include quantitative information regarding the skull of a patient, including skull volume (SV), skull density (SD), and skull area (SA). For example, AI module(s) can be configured to measure the mean SD of the skull of a patient by obtaining the mean attenuation value of pixels inside the segmented skull structure (see FIG. 5C) with attenuation values within 100-3000 HU (or 100-2000 HU). SV can be obtained by determining the quantity of pixels representing the patient's skull in multiple slices and multiplying the total quantity of pixels by a physical volume represented by each pixel. SA can be obtained from an axial slice wherein the axial slice contains the largest representation of the skull area of a patient.

In some embodiments, the set of quantitative metrics described in act 204 may include quantitative information regarding the ventricular spaces of a patient, including right ventricle volume (RVV), right ventricle density (RVD), left ventricle volume (LVV), left ventricle density (LVD), lateral ventricle volume(s), 3^{rd} ventricle volume (3VV), and 4^{th} ventricle volume (4VV). For example, AI module(s) can be configured to obtain a density measurement of one or more ventricles (i.e., RVD and/or LVD) by obtaining the mean attenuation value of pixels inside the corresponding segmented ventricular space(s) (see FIG. 5B) with attenuation values between −20-99 HU. A volume measurement of one or more of the patient's ventricular spaces (i.e., RVV, LVV, and/or 4VV) can be obtained by measuring the quantity of pixels inside the corresponding segmented ventricular space(s) (potentially across multiple slices) and scaling the quantity by a known volume value represented by each pixel. Other pieces of information regarding the ventricular spaces of a patient can be obtained by combining certain quantitative metrics. For example, biventricular volume (BVV) may be obtained by adding RVV and LVV.

In some embodiments, the set of quantitative metrics described in act 204 may include volume information associated with other structures, such as a volume of the hippocampus (and/or surrounding hippocampus), temporal lobe, amygdala, lateral sulcus, cerebellum white matter, cerebellum cortex, or central brainstem. Volume measurements may be obtained based on segmentations from multiple axial slices. In some implementations, the brain age of a patient (e.g., relative to their chronological age) can indicate a probability that the patient is experiencing a neurodegenerative disease. In some implementations, quantitative metrics described herein may be used to predict the presence of neurodegenerative diseases, such as Alzheimer's disease and/or others. For example, the volume of the hippocampus (and/or surrounding hippocampus), temporal lobe, amygdala, lateral sulcus, cerebellum white matter, cerebellum cortex, or central brainstem may be processed by one or more AI models (e.g., in combination with other patient-specific inputs, such as patient age and/or sex) to generate an output indicating the probability that a neurodegenerative disorder is present for a specific patient. The AI model(s) may be trained using a CT image dataset that includes images of patients experiencing target neurodegenerative disorders (e.g., Alzheimer's disease) and patients not experiencing such disorders. The AI model(s) can comprise regression models, decision tree models, random forest models, support vector machines, neural networks, or any other models/techniques described herein.

In some embodiments, the set of quantitative metrics described in act 204 may include quantitative information regarding the extra-axial CSF of a patient, including extra-axial CSF volume (EV) and extra-axial CSF density (ED). For example, AI module(s) can be configured to obtain EV based on the pixels within the segmented extra-axial CSF spaces (see FIG. 5E) (potentially across multiple image slices). ED can be obtained using pixels inside the corresponding segmented ventricular space(s) (see FIG. 5B) with attenuation values between −20-99 HU. Total CSF volume (TCSFV) may be obtained by adding EV, BVV, and 4VV.

Other quantitative metrics associated with the brain of the patient can be obtained using the systems and methods described herein and may be included in the set of quantitative metrics described in act 204. For example the set of quantitative metrics may include total intracranial volume (TIV), total intracranial area (TIA), right cerebral volume (RCV), fluid density right cerebral volume (fdRCV) (based on the volume that measures −20-20 HU), low density right cerebral volume (ldRCV) (based on the volume that measures 21-30 HU), intermediate density right cerebral volume (idRCV) (based on the volume that measures 31-40 HU), high density right cerebral volume (hdRCV) (based on the volume that measures 41-99 HU), right cerebral density (RCD) (based on the mean of pixels measuring −20-99 HU), right cerebral cortex volume (RCCV), left cerebral volume (LCV), fluid density left cerebral volume (fdLCV) (based on the volume that measures −20-20 HU)), low density left cerebral volume (IdLCV) (based on the volume that measures 21-30 HU), intermediate density left cerebral volume (idLCV) (based on the volume that measures 31-40 HU), high density left cerebral volume (hdLCV) (based on the volume that measures 41-99 HU), left cerebral density (LCD) (based on the mean of pixels measuring −20-99 HU), left cerebral cortex volume (LCCV), total cerebral volume (TCV) (e.g., RCV+LCV), fluid density total cerebral volume (fdTCV) (e.g., fdRCV+fdLCV), low density total cerebral volume (ldTCV) (e.g., ldRCV+ldLCV), intermediate density total cerebral volume (idTCV) (e.g., idRCV+idLCV), high density total cerebral volume (hdTCV) (e.g., hdRCV_hdLCV), total cerebral density (TCD) (e.g., mean of RCD+LCD), total cerebral cortex volume (TCCV) (e.g., RCCV+LCCV), cerebellar volume (CV), cerebellar density (CD) (based on the mean of pixels measuring −20-99 HU), fluid density cerebellar volume (fdCV) (based on the volume that measures −20-20 HU), low density cerebellar volume (ldCV) (based on the volume that measures 21-30 HU), intermediate density cerebellar volume (idCV) (based on the volume that measures 31-40 HU), high density cerebellar volume (hdCV) (based on the volume that measures 41-99 HU), brain stem volume (BSV), brain stem density (BSD) (based on the mean of pixels measuring −20-99 HU), fluid density brain stem volume (fdBSV) (based on the volume that measures −20-20 HU), low density brain stem volume (ldBSV) (based on the volume that measures 21-30 HU), intermediate density brain stem volume (idBSV) (based on the volume that measures 31-40 HU), high density brain stem volume (hdBSV) (based on the volume that measures 41-99 HU), supratentorial brain volume (SBV) (e.g., RCV+LCV), supratentorial brain density (SBD) (e.g., mean of RCD and LCD), total brain volume (TBV) (e.g., RCV+LCV+CV+BSV), total brain density (TBD) (e.g., mean of RCD, LCD, CD, and BSD), skull volume (SV), skull density (based on the mean of pixels measuring 100-3000 HU), right muscle volume (RMV), right muscle density (RMD) (based on the mean of pixels measuring −20-99 HU), left muscle volume (LMV), left muscle density (LMD) (based on the mean of pixels measuring −20-99 HU), total muscle volume (TMV) (e.g., RMV+LMV), total muscle density (TMD) (e.g., mean of RMD and LMD), and/or others.

The quantitative metrics listed hereinabove may be used to calculate key ratios related to the cognitive health of a patient, such as BVV:EV, BVV:TIV, EV:TIV, TCSFV:TIV, RVV:LVV, BVV:4VV, TBV:TIV, RCV:TIV, LCV:TIV, CV:TIV, BSV:TIV, TBV:TIV, TCSF:TBV, and/or others. Key ratios such as those listed hereinabove may be used to assess the overall cognitive health of a patient, diagnose one or more brain pathologies, and/or assign a brain age score to a patient.

In some instances, the quantitative metrics (or information based on the quantitative metrics) may be presented to the physician and/or patient. For example, the quantitative metrics may be presented as a pixel area (e.g., based on a quantity of image pixels within a region of a CT image, which may correspond to a physical area measurement), a pixel volume (e.g., based on quantities of image pixels within regions of multiple CT images, which may correspond to a physical volume measurement), a pixel density, a percentile score, a ratio, and/or other representations known in the art.

In some instances, the quantitative metrics may be used by a physician to diagnose a corresponding patient with one or more brain pathologies. For example, quantitative metrics that show abnormally enlarged ventricles and shrinkage of the cerebral cortex can indicate to a physician that the corresponding patient has dementia. Furthermore, the quantitative metrics can indicate an estimated likelihood of developing a particular brain pathology, wherein the estimated likelihood is represented as a percent likelihood of developing a particular brain pathology within a specified period of time (e.g., 25% chance of developing dementia in two years). Accordingly, an AI module can be trained to detect a brain pathology and/or provide an estimated likelihood of developing a particular brain pathology using quantitative metrics (e.g., obtained by CT imaging data processed in accordance with act 204).

The quantitative metrics and key ratios described hereinabove comprise pieces of information that may be extracted from head CT images using the methods and systems described in the present disclosure. Additional or alternative metrics can be obtained from head CT data and may be included in the set of quantitative metrics.

Act 206 of flow diagram 200 includes using the set of quantitative metrics referenced in act 204 to determine a brain age score of the brain of the patient. Brain age (or brain age score) may be calculated by using the set of quantitative metrics as input to a brain age estimation module configured to determine a brain age score of a patient. In some embodiments, the brain age estimation module may be a multivariate statistical model that assigns different weights to different quantitative metrics within the set of quantitative metrics used as input to the brain age estimation module. For example, where each of the quantitative metrics is represented as a percentile, the brain age estimation module may use the chronological age of a patient (e.g., 55 years) and the percentile of each quantitative metric (e.g., TBV: 40^{th} percentile, TBD: 40^{th} percentile, TCSFV: 40^{th} percentile, and atherosclerotic calcifications: 70^{th} percentile) and weight the chronological age and each percentile within a multivariate statistical model to obtain a brain age or brain age score (e.g., 65 years).

In some embodiments, the brain age estimation module can be one or more AI module(s) configured to determine a brain age score for a particular patient using the set of quantitative metrics described hereinabove with reference to act 204. For example, a brain age estimation module comprising an AI module(s) can use the set of quantitative metrics and the chronological age of a patient as input to determine a brain age score for the brain of the patient.

The AI module(s) may be trained using one or more sets of training data. For example, a set of training data for training the AI module(s) to provide a brain age score may include a plurality of training sets of quantitative metrics. Each training set of quantitative metrics of the plurality of training sets of quantitative metrics may be associated with a chronological age of a human patient and with one or more ground truth labels, such as a ground truth brain age score for each training set of quantitative metrics. Ground truth brain age scores may be assigned by human users (e.g., one or more physicians), using a multivariate statistical model (as noted above), and/or via other methods. One will appreciate that the set of training data may comprise any control input or additional input to facilitate configuring the AI module(s) to subsequently assign a brain age score to subsets of quantitative metrics.

A brain age score associated with the brain of a patient, as described hereinabove, may provide a physician with additional information regarding the cognitive health of a patient. For example, a brain age score can be used as a screening test for one or more underlying brain pathologies and/or used for a routine heath checkup. More specifically, medical providers may order a head CT scan to assess brain age to better understand the cognitive health of a patient or identify the underlying cause or severity of a particular disorder and/or pathology, including cardiovascular, metabolic, or neuropsychologic disorders and/or pathologies. In some instances, the brain age score may be used to screen healthy individuals (i.e., patients who are not experiencing symptoms of a particular brain pathology) for brain pathologies that have not yet manifested, providing physicians with an opportunity to detect pathologies early, possibly improving patient outcomes.

In some embodiments, the brain age score obtained in accordance with act 206 may be combined with other body composition metrics (e.g., atherosclerotic, carotid, coronary, or aortic calcifications, white matter hypodensities, heart size, emphysema severity, interstitial lung disease severity, liver fat or volume, kidney volume, splenic volume, visceral and subcutaneous abdominal fat, abdominal or chest muscle bulk or density, or bone density), physical testing results, neurophysical testing results, imaging exam findings (e.g., body CT or echocardiogram findings), and/or labs (e.g., cholesterol levels or lipid levels) to enhance the diagnostic and predictive capabilities of the systems and methods described herein. In some instances, the brain estimation module may adjust and/or weight the brain age score using information from the DICOM header or electronic medical record including patient age, gender, height, body weight, body mass index, and/or body surface area.

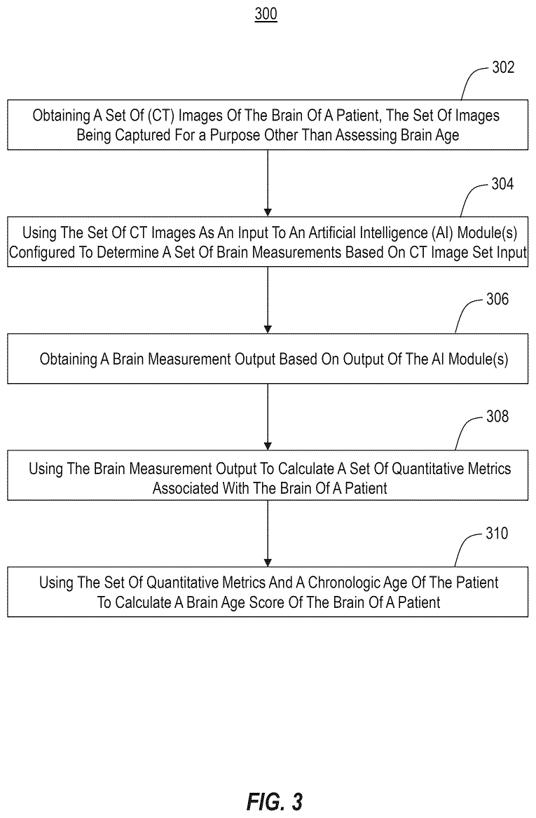

Attention is now directed to FIG. 3, which illustrates an example flow diagram 300. Flow diagram 300 is directed towards opportunistically screening for one or more brain pathologies, generating a brain age score, and/or assessing the cognitive health of a patient using CT imaging data. As described herein, opportunistic screening and/or assessment refers to passive screening for one or more particular brain pathologies and/or quantitative information about the cognitive health of a patient (including a brain age score) using medical imagery and/or reports obtained for a purpose that is independent of screening for the particular brain pathology and/or quantitative information. For example, a patient may receive a head CT after an accident resulting in a head injury, for the purpose of detecting a potential traumatic brain injury. Flow diagram 300 may be utilized to additionally screen for other brain pathologies and/or other quantitative information related to the brain of the patient. In some instances, preexisting sets of CT images may be processed in accordance with flow diagram 300 (i.e., CT images captured during a previous appointment and/or by a different physician). Furthermore, the acts depicted in flow diagram 300 may be used to screen healthy individuals (i.e., patients who are not experiencing symptoms of a particular brain pathology) for brain pathologies that have not yet manifested, providing physicians with an opportunity to detect pathologies early, possibly improving patient outcomes.

Act 302 of flow diagram 300 includes obtaining a set of CT images of the brain of a patient. The set of CT images may be similar to the set of CT images described in accordance with act 202 (see FIG. 2). The CT images described in act 302 are captured for a purpose other than assessing brain age. For example, the set of CT images may be captured after a patient experiences head trauma, headaches, impaired eyesight, impaired cognitive ability, or other reasons that typically result in a physician ordering a head CT exam for a patient. Accordingly, the set of CT images may be associated with electronic medical records (EMR) and may have been obtained through a previously performed head CT exam.

Act 304 of flow diagram 300 includes using the set of CT images as input to an AI module configured to determine a set of brain measurements based on the CT image set input. Each separate brain measurement of the set of brain measurements may be obtained using techniques similar to those described above with reference to act 204 of flow diagram 200 (see FIG. 2). For example, the AI module may be configured and/or trained to segment key brain structures represented in the set of CT images and provide measurements associated with the segmented key brain structures (e.g., see FIG. 5A-5E). In some embodiments, two or more AI modules can be used to perform act 304. For example, a pair of AI modules can operate in series, wherein a first AI module is configured to identify and segment key brain structures represented in the CT image set input and a second AI module is configured to determine brain measurements from the segmented CT image(s). Additionally, or alternatively, the AI module(s) may incorporate other image processing modules that are configured to determine brain measurements from the segmented CT image(s).

Act 306 of flow diagram 300 includes obtaining a brain measurement output based on output of the AI module(s). For example, AI module(s) may be configured to provide brain measurement output based on measurements taken of the brain of a patient represented in the CT image set input. The measurement(s) may be associated with various key brain structures of the brain of the patient represented in the CT image set input. For example, the measurement(s) may comprise area, density, volume, distance/length, and/or other measurements associated with a segmented brain structure (e.g., within the right ventricle) represented in the CT image set input (which may be determined by analyzing image pixels associated with the segment). In some instances, the measurements(s) may be determined using certain attenuation thresholds in order to target different structures inside the brain.

Accordingly, the AI module(s) may be configured to output one or more measurements in various forms (e.g., pixel volume, pixel density, distance/length, and/or others) of one or more key brain structures (e.g., the entire brain, the ventricular spaces, the sulci, the skull, atherosclerotic calcifications, extra axial CSF spaces, the cortex, white matter regions, grey matter regions, and/or others). In some implementations, brain measurement output is generated utilizing one or more additional module(s) for processing output of the AI module(s). In some instances, AI module(s) for analyzing one or more CT images can comprise multiple AI modules that are configured to determine different types of brain measurement output and/or quantitative metrics (e.g., one or more AI modules may be configured to provide brain measurement output related to the ventricular spaces, while one or more separate AI modules may be configured to provide brain measurement output related to the skull).

In some instances, different CT images within the CT image set input may provide largest measurements for different key brain structures. For example, one CT image may provide a largest representation of the total area of the skull (see FIG. 5C), whereas a separate CT image may provide a largest representation of the atherosclerotic calcifications (see FIG. 5D). In this regard, the subset of CT images identified by the AI module(s) may comprise any number of CT images (e.g., one or more), and the AI module(s) may obtain different measurements using different CT images of the subset of CT images (e.g., for providing brain measurement output).

Act 308 of flow diagram 300 includes using the brain measurement output to calculate a set of quantitative metrics associated with the brain of a patient. The set of quantitative metrics may be obtained using techniques similar to those described above with reference to act 204 of flow diagram 200. The set of quantitative metrics may correspond to the set quantitative metrics described above with reference to act 204.

Act 310 of flow diagram 300 includes using the set of quantitative metrics and a chronologic age of a patient to calculate a brain age score of the brain of a patient. The brain age score may be calculated using techniques similar to those described above with reference to act 206 of flow diagram 200. For example, the brain age score may be calculated using a brain age estimation module configured to estimate brain age based on quantitative metric set input.

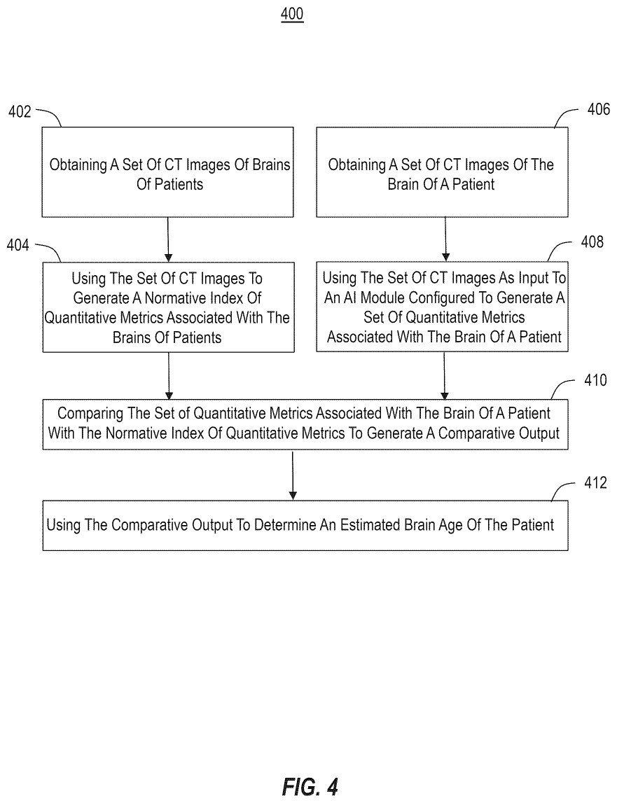

Attention is now directed to FIG. 4, which illustrates an example flow diagram 400. Flow diagram 400 is directed towards screening for one or more brain pathologies and/or assessing the cognitive health of a patient using a normative index of CT imaging data. The normative index is described herein as an index comprising head CT imaging data associated with heathy brains at different chronological ages. In some embodiments, the normative index comprises quantitative metrics (e.g., those described in accordance with act 204) associated with heathy brains at different chronological ages.

Act 402 of flow diagram 400 includes obtaining a set of CT images of brains of patients. The set of CT images obtained in accordance with act 402 may comprise multiple sets of CT images, and each set of CT images can be associated with a respective patient at a particular chronological age. Each set of CT images can capture a patient with a healthy brain for their chronological age (e.g., a brain not subjected to one or more pathologies that accelerate brain degradation). Sets of CT images that represent a range of chronological ages may be obtained in accordance with act 402. For instance, multiple sets of CT images representing patients with healthy brains may be obtained for each chronological age in a range of chronological ages (e.g., for each chronological age to be represented in the normative index of act 404). The set of CT images obtained in accordance with act 402 may be preexisting CT images (e.g., from one or more patient databases, research databases, hospital databases, etc.). For example, the set of CT images may be associated with EMR of one or more patients, wherein the CT images were obtained through a previously performed head CT exam for the patients.

Act 404 of flow diagram 400 includes generating a normative index of quantitative metrics associated with the brains of patients. The set of quantitative metrics can comprise a set of values for one or more brain age parameters, such as total brain volume, total brain density, ventricular volume, extra-axial cerebral spinal fluid (CSF) volume, atherosclerotic calcifications, and/or others. The quantitative metrics may be obtained using techniques similar to those described above with reference to act 204 of flow diagram 200 and/or acts 306, 308 of flow diagram 300 (see FIG. 2 and FIG. 3). For example, the set of CT images may be used as input to one or more AI modules configured to process the set of CT images to generate a set of quantitative metrics related to the brain of a patient (e.g., as conceptually represented in FIGS. 5A through 5E). The set of quantitative metrics may comprise any of the quantitative metrics described herein (e.g., with reference to act 204). Each set of quantitative metrics can be organized in the normative index according to patient chronological age. The normative index may comprise quantitative metrics for a range of chronological ages, such as quantitative metrics for (healthy) brains from 0 years to 100 years. For each chronological age represented in the normative index, the applicable quantitative metrics can be derived from CT imagery of multiple patients with health brains at the chronological age.

The normative index generated according to act 404 can be used to facilitate brain age estimation for patients based on CT imagery of the patient's brain. For instance, act 406 of flow diagram 400 includes obtaining a set of CT images of the brain of a patient. The set of CT images may be similar to the set of CT images described in accordance with act 202 (see FIG. 2). In some embodiments, the CT images described in act 406 are captured for a purpose other than assessing brain age. For example, the set of CT images may be captured after a patient experiences head trauma, headaches, impaired eyesight, impaired cognitive ability, or other reasons that typically result in a physician ordering a head CT exam for a patient. The set of CT images may be associated with electronic medical records (EMR) and may have been obtained through a previously performed head CT exam.

Act 408 of flow diagram 400 includes using the set of CT images as an input to an artificial intelligence (AI) module configured to process the set of CT images to generate a set of quantitative metrics associated with the brain of the patient referenced in act 406. The quantitative metrics may be obtained using techniques similar to those described above with reference to act 204 of flow diagram 200 and acts 306, 308 of flow diagram 300 (see FIG. 2 and FIG. 3). For example, the set of CT images may be used as input to an AI module configured to process the set of CT images to generate a set of quantitative metrics related to the brain of a patient (e.g., similar to FIGS. 5A through 5E). The set of quantitative metrics may comprise any of the quantitative metrics described herein.

Act 410 of flow diagram 400 includes comparing the set of quantitative metrics associated with the brain of the patient referenced in act 408 with the normative index of quantitative metrics referenced in act 404 to generate a comparative output. The comparative output may comprise a statistical comparison of the quantitative metrics associated with the brain of the patient with the normative values included in the comparative index. For example, a comparative output may indicate that the brain of a current patient at a particular chronological age (e.g., 55 years) is most similar to a heathy brain at a different chronological age (e.g., 65 years).

Generating the comparative output may involve using the set of quantitative metrics referenced in act 408 as input to a brain age comparison module configured to generate a similarity measure the between the set of quantitative metrics referenced in act 408 and normative values associated with multiple chronological ages represented in the normative index. The similarity measure may comprise a composite or overall similarity measure and/or may comprise one or more similarity components (e.g., with different similarity components being determined for each of the quantitative metrics being compared). The similarity measure may weight different quantitative metrics differently, in some implementations. Various types of similarity measures are within the scope of the present disclosure, such as difference metrics, distance-based similarity (e.g., Euclidean, Hamming, Mahalanobis), correlation-based similarity, cosine similarity, and/or others. In some implementations, the similarity measure comprises a similarity score output by one or more AI modules (e.g., using vector embeddings). Comparing a single input set of quantitative metrics for a single patient to the normative index in accordance with act 410 can result in a comparative output comprising a set of similarity scores, which can include a separate similarity score indicating similarity between the single input set of quantitative metrics and the quantitative metrics associated with a range of chronological ages represented in the normative index. The range can comprise the entire range of chronological ages represented in the normative index, or can comprise a subset range from the entire range (e.g., based on the chronological age of the single patient for whom brain age is being estimated). In some instances, the comparative output can be based on other patient attributes/characteristics, such as gender, body composition metrics, physiologic measurements, etc.

Act 412 of flow diagram 400 includes using the comparative output to determine an estimated brain age of the patient. For instance, where the comparative output comprises similarity scores between the input set of quantitative metrics and the quantitative metrics for a range of chronological ages represented in the normative index, the estimated brain age of the patient may be determined to be the chronological age associated with the highest similarity score from the comparative output.

The brain age estimation generated by the brain age estimation module may provide a physician with additional information regarding the cognitive health of a patient. For example, a brain age score can be used as a screening test for one or more underlying brain pathologies and/or used for a routine heath checkup. More specifically, medical providers may order a head CT scan to assess brain age to better understand the cognitive health of a patient or identify the underlying cause or severity of a particular disorder and/or pathology, including cardiovascular, metabolic, or neuropsychologic disorders and/or pathologies. In some instances, the brain age estimation may be used to screen healthy individuals (i.e., patients who are not experiencing symptoms of a particular brain pathology) for brain pathologies that have not yet manifested, providing physicians with an opportunity to detect pathologies early, possibly improving patient outcomes.

In some embodiments, the brain age estimation obtained in accordance with act 412 may be combined with other body composition metrics (e.g., atherosclerotic, carotid, coronary, or aortic calcifications, white matter hypodensities, heart size, emphysema severity, interstitial lung disease severity, liver fat or volume, kidney volume, splenic volume, visceral and subcutaneous abdominal fat, abdominal or chest muscle bulk or density, or bone density), physical testing results, neurophysical testing results, imaging exam findings (e.g., body CT or echocardiogram findings), and/or labs (e.g., cholesterol levels or lipid levels) to enhance the diagnostic and predictive capabilities of the systems and methods described herein. In some instances, the brain estimation module may adjust and/or weight the brain age score using information from the DICOM header or electronic medical record including patient age, gender, height, body weight, body mass index, and/or body surface area.

The results of the systems and methods described hereinabove may be operable to trigger additional action associated with the patient. In particular, the additional action associated with the patient may comprise providing a notification to one or more relevant entities. A relevant entity may comprise, for example, the patient, a legal guardian of the patient, a primary care or other physician of the patient, and/or others. The notification may be configured to apprise the one or more relevant entities of the patient's cognitive health outlook (e.g., a notification of a detected brain pathology, and/or a brain age score) thereby enabling the patient to seek medical attention as appropriate for diagnosis and/or treatment of one or more brain pathologies and/or other cognitive health issues.

The notification provided to the relevant entity may take on various forms and/or may be provided in various ways. For example, in some embodiments, the notification takes the form of a report generated for viewing by the one or more relevant entities. In some instances, the report includes one or more representations of the patient's brain (e.g., described hereinabove with reference to FIG. 5A through FIG. 5E. Additionally, or alternatively, the report can include a set of quantitative metrics generated as described hereinabove with reference to act 204 of flow diagram 200, acts 306, 308 of flow diagram 300, and/or act 408 of flow diagram 400.

Furthermore, in some instances, the report includes a representation of one or more CT images of the subset of CT images identified by the AI module as described hereinabove with reference to act 204. The representation of the one or more CT images may be labeled in a manner that emphasizes the key brain structure and associated quantitative metrics. For example, one or more representations of the key brain structures may be displayed over the one or more CT images, such as a highlighted segment showing area, one or more colored lines showing segmented structures, etc. A report may comprise any additional or alternative graphics, charts, images, quantitative metrics, and/or information related to the patient not explicitly described herein.

In some instances, such as where a patient is classified as having a brain pathology or is at high risk to develop a brain pathology, a report may comprise further action suggested for the patient (e.g., by providing contact information for a neurologist or other medical practitioner or by providing a selectable link for initiating care with a medical practitioner).

In some instances, a report is automatically generated and transmitted to the relevant entity, whereas, in other instances, the report is transmitted to an intermediate entity (e.g., a reviewing physician or other medical practitioner) for review before transmitting to another entity. A report may be transmitted to the relevant entity through any suitable means, such as via e-mail, a printed document, text message, through a user-interactable interface (e.g., a patient portal), and/or other means.

In some instances, the reports from one or more patients may be entered into a digital patient tracking system. The patient tracking system could incorporate patient data from the output report as well as patient data from the EMR. The patient tracking system could provide a mechanism to follow up patient progress with referrals to clinical providers, including, for example, an ability to “snooze” a patient, putting their progress on hold temporarily but triggering follow up at a future time point.

In some instances, the information provided in the report may be added to or used to update the normative index as described hereinabove with reference to acts 402 and 404 of flow diagram 400.

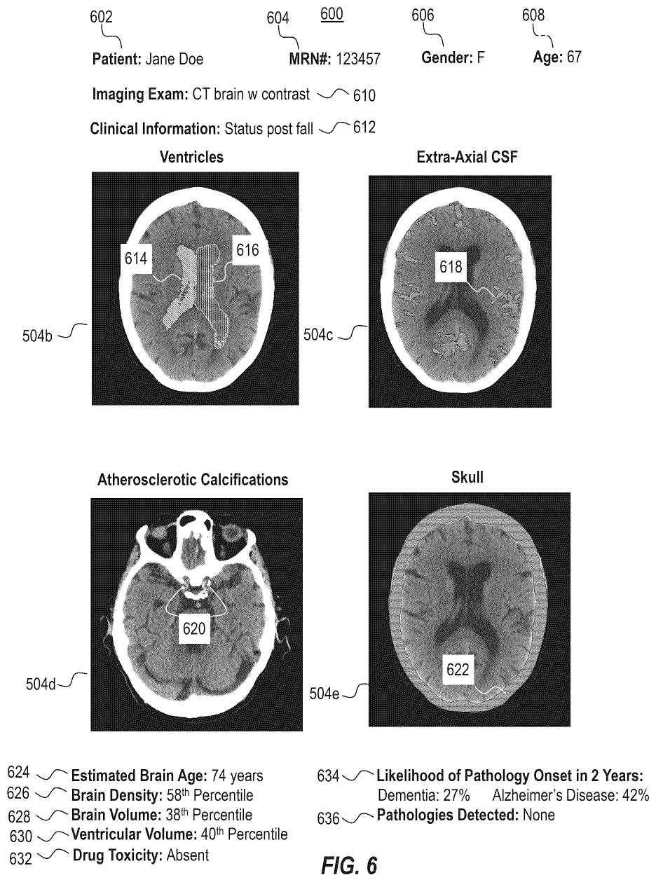

By way of illustration, FIG. 6 illustrates example components of a report 600. For example, the report 600 includes patient information, such as the patient's name 602, medical record number 604, age 606, gender 608, type of imaging exam 610 (“CT brain with contrast” in the example of FIG. 6), and/or clinical information 612 related to the patient and/or the CT imaging performed (“Status post fall” in the example of FIG. 6). In the example of FIG. 6, the report 600 includes one or more CT images (e.g., images 504b-504d), wherein the CT images depict key brain structures such as a right ventricle area 614, a left ventricle area 616, an extra-axial CSF area 618, atherosclerotic calcifications 620, and a skull area 622. The right ventricle area 614, left ventricle area 616, extra-axial CSF area 618, atherosclerotic calcifications 620, skull area 622, and/or other measurements described hereinabove may be generated utilizing one or more AI modules, as discussed hereinabove with reference to act 204 of flow diagram 200, acts 306,308 of flow diagram 300, and/or act 408 of flow diagram 400.

The report 600 may further comprise an estimated brain age and/or brain age score 624. The estimated brain age and/or brain age score 624 may be generated utilizing one or more brain age estimation modules, as discussed hereinabove with reference to act 206 of flow diagram 200, act 310 of flow diagram 300, and/or act 412 of flow diagram 400. The report 600 may further comprise a set of quantitative metrics related to key brain structures, such as brain density 626, brain volume 628, and/or ventricular volume 630. Other quantitative metrics described hereinabove may be additionally or alternatively included. In the illustrated example, the quantitative metrics are represented by percentile rankings (although other methods for representing the quantitative metrics are within the scope of the present disclosure, as discussed above). The quantitative metrics may be generated utilizing one or more AI modules, as discussed hereinabove with reference to act 204 of flow diagram 200, acts 306, 308 of flow diagram 300, and/or act 408 of flow diagram 400.

As discussed hereinabove, multiple brain pathologies (e.g., dementia, Alzheimer's disease, stroke, hydrocephalus, drug toxicity and/or others) may be detected using the brain age estimation/brain age score and/or the set of quantitative metrics. Furthermore, the likelihood of pathology onset may be calculated using the brain age estimation/brain age score and/or the set of quantitative metrics. Accordingly, the report 600 may comprise a notification of detected drug toxicity 632, a notification of detected pathologies 636, and an estimation of pathology onset 634. The detection of multiple pathologies as well as the estimated likelihood of pathology onset may be generated using the systems and methods described hereinabove.

### Additional Details Related to Implementing the Disclosed Embodiments

The principles disclosed herein may be implemented in various formats. For example, the various techniques discussed herein may be performed as a method that includes various acts for achieving particular results or benefits. In some instances, the techniques discussed herein are represented in computer-executable instructions that may be stored on one or more hardware storage devices. The computer-executable instructions may be executable by one or more processors to carry out (or to configure a system to carry out) the disclosed techniques. In some embodiments, a system may be configured to send the computer-executable instructions to a remote device to configure the remote device for carrying out the disclosed techniques.

Disclosed embodiments may comprise or utilize a special purpose or general-purpose computer including computer hardware, as discussed hereinabove. Disclosed embodiments also include physical and other computer-readable media for carrying or storing computer-executable instructions and/or data structures. Such computer-readable media can be any available media that can be accessed by a general purpose or special purpose computer system.

Computer-readable media that store computer-executable instructions are physical storage media. Computer-readable media that carry computer-executable instructions are transmission media. Thus, by way of example, and not limitation, embodiments of the invention can comprise at least two distinctly different kinds of computer-readable media: physical computer-readable storage media (e.g., hardware storage devices) and transmission computer-readable media.

Physical computer-readable storage media includes hardware storage devices such as RAM, ROM, EEPROM, CD-ROM or other optical disk storage (such as CDs, DVDs, etc.), magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmission media can include a network and/or data links which can be used to carry program code in the form of computer-executable instructions or data structures and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general-purpose computer, special purpose computer, or special purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Disclosed embodiments may comprise or utilize cloud computing. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, wearable devices, and the like. The invention may also be practiced in distributed system environments where multiple computer systems (e.g., local and remote systems), which are linked through a network (either by hardwired data links, wireless data links, or by a combination of hardwired and wireless data links), perform tasks. In a distributed system environment, program modules may be located in local and/or remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Application-specific Standard Products (ASSPs), System-on-a-chip systems (SOCs), Complex Programmable Logic Devices (CPLDs), central processing units (CPUs), graphics processing units (GPUs), and/or others.

As used herein, the terms “executable module,” “executable component,” “component,” “module,” or “engine” can refer to hardware processing units or to software objects, routines, or methods that may be executed on one or more computer systems. The different components, modules, engines, and services described herein may be implemented as objects or processors that execute on one or more computer systems (e.g., as separate threads).

### CONCLUSION

Although the subject matter described herein is provided in language specific to structural features and/or methodological acts, it is to be understood that the subject matter defined in the appended claims is not necessarily limited to the described features or acts so described. Rather, the described features and acts are disclosed as example forms of implementing the claims.

Unless defined otherwise, all technical and scientific terms used herein have the same meaning as commonly understood by one of ordinary skill in the art to which the present disclosure pertains.

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

It will also be appreciated that systems and methods according to certain embodiments of the present disclosure may include, incorporate, or otherwise comprise properties or features (e.g., components, members, elements, parts, and/or portions) described in other embodiments. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment unless so stated. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

What is currently claimed is:

1. A computer-implemented method for assessing brain age, the computer-implemented method comprising:
obtaining a set of computed tomography (CT) images, the set of CT images capturing at least a portion of a brain of a patient;
processing the set of CT images using one or more artificial intelligence (AI) modules to obtain a set of quantitative metrics associated with the brain of the patient, the set of quantitative metrics comprising:
brain volume,
ventricular volume,
cerebral spinal fluid (CSF) volume, and/or
atherosclerotic calcifications; and
using the set of quantitative metrics and a chronological age of the patient as input to a brain age estimation module to determine a brain age score of the brain of the patient.

2. The computer-implemented method of claim 1, wherein the set of quantitative metrics comprises: hippocampus volume, temporal lobe volume, amygdala volume, lateral ventricles volume, 3^{rd} ventricle volume, 4^{th} ventricle volume, lateral sulcus volume, cerebellum white matter volume, cerebellum cortex volume, cerebral brainstem volume, surrounding hippocampus volume, skull bone density, or total brain density.

3. The computer-implemented method of claim 2, further comprising using the set of quantitative metrics to determine a probability of one or more neurodegenerative diseases.

4. The computer-implemented method of claim 1, wherein the brain age score is further based on one or more body composition metrics, physiological measurements, cognitive testing results, physical testing results, neuropsychologic testing results, other image exam findings, or lab results.

5. A computer-implemented method for assessing brain age, the computer-implemented method comprising:
obtaining a set of computed tomography (CT) images, the set of CT images capturing at least a portion of a brain of a patient, the set of CT images being captured for a purpose independent of assessing brain age;
using the set of CT images as an input to an artificial intelligence (AI) module configured to determine a brain measurement based on CT image set input;
obtaining a brain measurement output based on output of the AI module;
using the brain measurement output to calculate a set of quantitative metrics associated with the brain of the patient; and
using the set of quantitative metrics and a chronologic age of the patient to calculate a brain age score of the brain of the patient.

6. The computer-implemented method of claim 5, wherein the set of CT images was obtained as part of clinical care, screening, or research.

7. The computer-implemented method of claim 5, the set of CT images being a nonenhanced CT exam.

8. The computer-implemented method of claim 5, wherein the AI module comprises a machine learning module configured to:
identify a subset of CT images from the set of CT images, the subset of CT images comprising one or more representations of one or more key brain structures; and
process the subset of CT images to determine the brain measurement output.

9. The computer-implemented method of claim 8, wherein the machine learning module is a deep learning module.

10. The computer-implemented method of claim 8, the machine learning module being trained using training data comprising:
a plurality of training sets of CT images; and
for each training set of CT images of the plurality of training sets of CT images: an identification of a respective subset of CT images, and a respective brain measurement based on the respective subset of CT images.

11. The computer-implemented method of claim 5, wherein the set of quantitative metrics comprises one or more of: total brain volume, total brain density, ventricular volume, cerebral spinal fluid (CSF) volume, atherosclerotic calcifications, and skull bone density.

12. The computer-implemented method of claim 5, further comprising:
using the set of quantitative metrics to screen for brain pathologies.

13. The computer-implemented method of claim 12, further comprising:
after detecting a brain pathology based on the set of quantitative metrics, providing a notification to one or more relevant entities.

14. The computer-implemented method of claim 5, wherein the set of CT images is restricted to images with mean attenuation values of −10 HU to +99 HU.

15. The computer-implemented method of claim 5, wherein the set of CT images is restricted to images capturing a skull of the patient and that omit soft tissue structures outside the skull.

16. A computer-implemented method for opportunistic assessment of brain age, the computer-implemented method comprising:
obtaining a set of computed tomography (CT) images, the set of CT images capturing at least a portion of a brain of a patient, the set of CT images being captured for a purpose independent of assessing brain age;
using the set of CT images as an input to an artificial intelligence (AI) module configured to generate a set of quantitative metrics associated with the brain of the patient, wherein the set of quantitative metrics comprises a set of values for one or more brain age parameters, the one or more brain age parameters comprising one or more of: total brain volume, total brain density, ventricular volume, extra-axial cerebral spinal fluid (CSF) volume, or atherosclerotic calcifications;
compare the set of quantitative metrics to a normative index of head CT data to determine a comparative output, the normative index comprising a set of normative values for the one or more brain age parameters, wherein the set of normative values is associated with healthy brains at different chronological ages; and
using the comparative output to calculate an estimated brain age of the brain of the patient.

17. The computer-implemented method of claim 16, wherein the AI module comprises a machine learning module configured to:
identify a subset of CT images from the set of CT images, the subset of CT images comprising one or more representations of one or more key brain structures; and
use the subset of CT images to generate a set of quantitative metrics associated with the brain of the patient.

18. The computer-implemented method of claim 17, the machine learning module being trained using training data comprising:
a plurality of training sets of CT images; and
for each training set of CT images of the plurality of training sets of CT images: an identification of a respective subset of CT images, and a respective measurement for the one or more brain age parameters.

19. The computer-implemented method of claim 16, wherein the normative index further comprises a set of normative CT images associated with different chronological ages.

20. The computer-implemented method of claim 19, wherein the set of normative values of the normative index of head CT data one or more AI modules based on the set of normative CT images.
